(19) United States
(12) Patent Application Publication
(10) Pub. No.: US 2006/0148677 A1
(43) Pub. Date: Jul. 6, 2006

(54) Chondrogenesis stimulator

(75) Inventors: Yukio Kato, Hiroshima (JP); Katsumi Fujimoto, Hiroshima (JP)

Correspondence Address: BROWDY AND NEIMARK, P.L.L.C.;624 NINTH STREET, NW, SUITE 300, WASHINGTON, DC, 20001-5303, US

(73) Assignee: CHUGAI SEIYAKU KABUSHIKI KAISHA, TOKYO (JP)

(21) Appl. No.: 11/304,595

(22) Filed: Dec. 16, 2005

Related U.S. Application Data
(63) Continuation of application No. 10/049,957, filed on Feb. 19, 2002, now abandoned.

(30) Foreign Application Priority Data
Aug. 19, 1999 (JP) 232966/1999

(51) Int. Cl. A61K 38/40

(52) U.S. Cl. 514/6

(57) ABSTRACT

There are provided a chondrogenesis stimulator containing MTf, a chondrogenic differentiation suppressing agent containing an MTf antagonist, a screening method for obtaining an MTf activating agent, an MTf activating agent obtained by the screening method, a chondrogenesis stimulator containing an MTf activating agent as obtained by the screening method, and MTf which lacks the GPI anchor region.

### CROSS-REFERENCE TO RELATED APPLICATIONS

The present application is a continuation of copending parent application Ser. No. 10/049,957, nationalized on Feb. 19, 2002, which application was the national stage under 35 U.S.C. 371 of PCT/JP00/05590, filed Aug. 21, 2000, and claiming priority from Japanese application 232966/1999 filed Aug. 19, 1999.

### TECHNICAL FIELD

This invention relates to a novel chondrogenesis stimulator. More specifically, the invention relates to a chondrogenesis stimulator containing a membrane-bound transferrin-like protein (hereunder sometimes referred to as MTf).

### PRIOR ART

The cartilage tissue of animals is composed of chondrocytes and matrix. The cartilage tissue accounts for the greater part of the skeleton at the prenatal stage and it is postnatally replaced with bone tissue due to endochondral ossification. When endochondral ossification starts, chondrocytes change from the resting to proliferating phase and the proliferating chondrocytes are then differentiated into hypertrophic chondrocytes [Reference; “Hone no kagaku (Science of Bone)”, ed. by Tsuda et al., pp. 11-29, Tokyo Ishiyaku Shuppan, 1982]. Thus, it has been well known that chondrocytes are essential cells for the formation of bone tissue, particularly at the growth stage. However, the differentiation of chondrocytes and the endochondral ossification remain unknown in many aspects.

The cell membrane of chondrocytes has characteristic glycoproteins and their membrane proteins might contribute to the unique features of chondrocytes that distinguish them from the cells of other connective tissues (as exemplified by spherical cell morphology, massive secretion of cartilage matrix, survival and proliferation in soft agar). Based on this hypothesis, Yan et al. (Yan et al.; J. Biol. Chem., vol. 265, pp. 10125-10131, 1990) and Kato et al. (Kato et al., Journal of the Society of Bone Metabolism of Japan, vol. 10, No. 2, pp. 187-192, 1992) investigated the effects of various lectins on the differentiation and proliferation of chondrocytes and, among other things, they have shown that concanavalin A (hereunder sometimes referred to as Con A) which is Jack bean lectin and which has affinity for α-D-mannose residue and α-D-glucose residue is a potent stimulator of chondrogenic differentiation, with the increase in proteoglycan synthesis being one of the criteria for the Con A activity. Chondrocytes treated with Con A change their shape from the immature flat morphology to the differentiated spherical form, inducing the production of proteoglycan and type II collagen which are markers of chondrogenic differentiation, the expression of alkaline phosphatase, etc., and even the calcification. Other lectins do not exert such differentiation inducing action.

In an attempt to search for a receptor mediating the action of Con A, Kawamoto et al. (Kawamoto et al., Eur. J. Biochem. vol. 256, pp. 503-509, 1998) paid particular attention to a protein of 76 kDa (p76) which was one of the about 20 kinds of Con A-binding proteins on chondrocytes and which would be expressed at lower levels in retinoic acid treated chondrocytes (upon treatment with retinoic acid, chondrocytes are dedifferentiated to lose reactivity with Con A). After purifying p76 from the plasma membrane fraction of rabbit chondrocytes by Con A affinity column chromatography, the N-terminal amino acid sequence was determined and the gene was cloned. In view of the determined amino acid sequence and the nucleic acid sequence of its cDNA, p76 showed 86% amino acid identity with melanotransferrin (p97) and was considered its counterpart; p97 is known as a tumor-associated antigen expressed at high levels in human tumors such as melanoma. The physiological functions of p97 are yet to be known and its expression has been reported to be high in only tumor cells, with very low detectability in normal tissue.

In view of its ability to bind with Con A, p76 is presumably involved in the differentiation of chondrocytes or in the development of their function; however, nothing has been confirmed about the effects this protein would actually impose on chondrocytes or their precursors.

An object, therefore, of the present invention is to identify a substance that will be involved in the differentiation of chondrocytes and provide a novel chondrogenesis stimulator using the substance. The present invention will lead to the invention of a substance that can control the function of chondrocytes and which eventually enables promoted osteogenesis. The substance can potentially lead to the treatment, prevention and diagnosis of new types of diseases associated with the cartilage and bone metabolisms.

### DISCLOSURE OF THE INVENTION

In order to attain the stated object, the present inventors made intensive studies and found that differentiation to cartilage could be markedly induced by overexpressing a membrane-bound transferrin-like protein (MTf)-gene in mouse cell line ATDC5 which retained the ability to differentiate to chondrocytes but which would hardly differentiate in the absence of stimulation.

Thus, the present invention provides a chondrogenesis stimulator containing a membrane-bound transferrin-like protein (MTf).

The MTf is preferably rabbit p76 protein, human p97 protein, mouse MTf protein, as well as a protein demonstrating the MTf activity that has an amino acid sequence encoded by DNA which hybridizes, under stringent conditions, with a DNA that encodes p76 protein or p97 protein or mouse MTf, and human p97 protein is particularly preferred.

The MTf is most preferably selected from the following:

1) a protein having the amino acid sequence of SEQ ID NO: 2;

2) a protein having the amino acid sequence of SEQ ID NO: 4;

3) a protein having the amino acid sequence of SEQ ID NO: 15; and

4) a protein demonstrating the MTf activity that has an amino acid sequence encoded by DNA which hybridizes, under stringent conditions, with a DNA encoding the protein of SEQ ID NO: 2, 4 or 15.

The present invention also provides said chondrogenesis stimulator in which the MTf lacks the GPI anchor region.

The chondrogenesis stimulator of the invention becomes more effective when used in combination with an MTf activating agent and/or insulin.

The chondrogenesis stimulator of the invention is useful with the following diseases: OA (osteoarthritis); RA (rheumatoid arthritis); injury of articular cartilage due to trauma; maintenance of chondrocyte phenotype in autologous transplantation of chondrocytes; reconstruction of cartilage in the ear, trachea or nose; osteochondritis dissecans; regeneration of intervetebral disk or meniscus; fractured bone; and osteogenesis from cartilage.

The invention further provides an agent for gene therapy to promote chondrogenesis which contains as an active ingredient an expression vector incorporating a DNA coding for any one of the following proteins:

1) a protein having the amino acid sequence of SEQ ID NO: 2;

2) a protein having the amino acid sequence of SEQ ID NO: 4;

3) a protein having the amino acid sequence of SEQ ID NO: 15; 4) a protein demonstrating the MTf activity that has an amino acid sequence encoded by DNA which hybridizes, under stringent conditions, with a DNA encoding the protein of SEQ ID NO: 2, 4 or 15; and

5) a protein which is the same as protein 1), 2), 3) or 4), except that it lacks the GPI anchor region.

The present invention further provides a chondrogenic differentiation suppressing agent containing an MTf antagonist.

The MTf antagonist is preferably an anti-MTf antibody or an oligonucleotide or an oligonucleotide analog that are hybridizable with a nucleic acid encoding MTf.

The present invention further provides a method for screening an MTf activating agent which comprises the steps of:

1) preparing a cell line in which MTf is overexpressed, wherein said cell line retains the ability to differentiate to chondrocytes but hardly differentiate without stimulation;

2) adding candidate substances to the cell line prepared in step 1) and culturing it for a specified period of time; and

3) examining the cell line for induced chondrogenic differentiation and selecting an MTf activating agent from the candidate substances.

The present invention also provides an MTf activating agent as obtained by the method described above.

The present invention also provides a chondrogenesis stimulator containing an MTf activating agent as obtained by the method described above.

The present invention further provides MTf which lacks the GPI anchor region.

### BRIEF DESCRIPTION OF THE DRAWINGS

FIG. 1 shows a scheme of the procedure of preparing an MTf overexpressing ATDC5 variant cells;

FIG. 2 shows the expression of the MTf gene in the variant of ATDC5 cells as analyzed by Northern blotting (photographs of electrophoresis);

FIG. 3 shows the expression of the MTf protein in the variant of ATDC5 cells as analyzed by Western blotting (photographs of electrophoresis);

FIG. 4 is a set of photographs showing that MTf overexpressing cell lines (Full-1 and Full-5) demonstrate the morphology of differentiated chondrocytes in comparison with control cells (pC-1), all of which are cultured for 29 days in the absence of insulin (biological morphology is shown);

FIG. 5 is a set of photographs showing that MTf overexpressing cell lines (Full-1 and Full-5) demonstrate the morphology of differentiated chondrocytes in comparison with control cells (pC-1), all of which are cultured for 29 days in the presence of insulin (biological morphology is shown);

FIG. 6 is a set of photographs showing the effects of the addition of the conditioned medium of rabbit chondrocytes on the induction of chondrogenic differentiation (biological morphology is shown); and

FIG. 7 shows the result of RT-PCR Southern blotting which demonstrates the overexpression of antisense MTf RNA and the suppression of aggrecan synthesis in the presence and absence of insulin.

### BEST MODE FOR CARRYING OUT THE INVENTION

The term “membrane-bound transferrin-like protein (MTf)” as used in the invention means a protein on the cell membrane of chondrocytes that binds to Con A and which has iron-binding sites as transferrin does. Preferably, the term means a protein having the ability to mediate the induction of chondrogenic differentiation by Con A.

The term MTf has conventionally been used as the abbreviation for melanotransferrin (p97) known as a tumor antigen expressed at high levels in melanoma and other tumors. As it turned out, however, p97 is also expressed at high levels in tissues other than cancer, particularly in cartilage. Since p97 is by no means specific to cancer tissue, the present inventors redefined the term MTf as meaning “membrane-bound transferrin-like protein”.

The term “MTf activity” as used herein means an activity that induces undifferentiated cells to differentiate to cartilage and which promotes chondrocytes to develop their function.

Examples of MTf include but are not limited to rabbit p76 protein, p97 protein which is a human protein homologous to rabbit p76 protein, mouse MTf protein, as well as proteins having MTf activity that contain alterations such as deletion, substitution or addition of one or more of the amino acids of these proteins, and proteins having MTf activity amino acid sequences encoded by DNA which hybridizes with DNA encoding p76 protein or p97 protein or mouse MTf protein under stringent conditions [a typical method is descried in Molecular Cloning: A Laboratory Manual, Sambrook et al., Cold Spring Harbor Laboratory Press, 1989 and consists, for example, of hybridization at 68° C. in a solution containing 6×SSC, 0.5% SDS, 10 mM EDTA, 5× Denhardt's solution and 10 mg/ml of denatured salmon sperm DNA].

Rabbit p76 protein is homologous to human p97 protein and sometimes called rabbit p97 (Kawamoto et al., Eur. J. Biochem. vol. 256, pp. 503-509, 1998). The nucleotide and amino acid sequences of rabbit p76 protein are identified by SEQ ID NO: 1 and SEQ ID NO: 2, respectively. The nucleotide and amino acid sequences of human p97 protein are also known (Rose, T. M. et al., Pro NAS 83, 1261-1265, 1986). The nucleotide and amino acid sequences of human p97 protein are identified by SEQ ID NO: 3 and SEQ ID NO: 4, respectively. Mouse MTf protein is described in Biochim. Biophys. Acta, 1447:258-264, 1999 and its nucleotide and amino sequences are identified by SEQ ID NO: 14 and SEQ ID NO: 15, respectively. The homology between the MTf proteins over animal species are high and the amino acid identity is 83% between mouse and human, 82% between mouse and rabbit, and 86% between human and rabbit.

p76/p97 proteins are GPI anchored proteins which have glycolipid GPI (glycosylphosphatidylinositol) bound to the carboxyl group in C-terminal amino acid so that they are bound to membranes using GPI as an anchor (for p76, see Ryo Oda, Journal of Dentistry, Hiroshima University, vol. 29, No. 1, pp. 40-57, 1997; for p97, see Alemany, R. et al., J. Cell Science, 104, 1155-1162, 1993). As will be shown later in the Examples, it was verified that not only full-length MTf but also GPI anchor lacking MTf or soluble MTf have a chondrogenic differentiation inducing activity when they were expressed in non-MTf-expressing cells. Therefore, such soluble MTf, preferably the GPI anchor lacking MTf, can also be used as a chondrogenesis regulating agent. The GPI anchor lacking MTf as used herein means a soluble MTf which lacks all or part of the GPI anchor moiety; in the case of rabbit MTf, it may be exemplified by MTf in which the 28 residues at C-terminal necessary for GPI anchor binding are deleted and in the case of human MTf and mouse MTf, it may be exemplified by MTf in which the 30 residues at C-terminal necessary for GPI anchor binding are deleted.

The MTf to be used in the invention may be of a native or recombinant form and either form can be obtained by methods known in the art. The respective types of MTf are illustrated below.

Native Form

MTf can be prepared by the method described in JP 7-82297A using chondrocytes. Briefly, cartilage tissues of various animals can be used as chondrocyte source; for example, a rabbit costal growth plate cartilage as the source is treated with protease and collagenase in accordance with the method of Kato et al. (Kato et al.; J. Cell Biol., vol. 100, pp. 477-485, 1985) to obtain chondrocytes. The isolated chondrocytes can be incubated in a medium containing fetal calf serum (FCS) on a culture dish at 37° C. in an atmosphere of 5% CO₂ and 95% air. The cultured chondrocytes are recovered, homogenized with a homogenizer and subjected to sedimentation equilibrium centrifugation by 17%/40% sucrose equilibrium density gradient to separate membrane proteins. The obtained membrane protein fraction is directly subjected on a concanavalin A affinity column; alternatively, in order to remove membrane proteins that bind to lectins other than concanavalin A, the membrane protein fraction is first subjected on an affinity column of wheat germ lectin which is a typical lectin and then subjected on a concanavalin A affinity column. By these and other techniques, more of the concanavalin A binding protein fraction can be separated. The specificity of the obtained concanavalin A bound protein fractions for chondrocytes can be evaluated by comparing these fractions through SDS-polyacrylamide gel electrophoresis (SDS-PAGE). After identifying the desired chondrocyte specific glycoproteins, bands of interest are excised from the gel, extracted and purified by electroelution or other suitable techniques. The resulting glycoproteins can be analyzed for the sugar chains after excising by endoglicosidase.

Recombinant Form

Recombinant MTf can be prepared by the methods described in the Examples of the invention or modifications thereof; by these methods, plasmids incorporating the MTf gene are transfected to host cells for expressing the MTf protein.

However, these are not the only methods that can be used and various methods of transformation and various host cells that are known in the art can also be used. For example, a gene encoding MTf may be inserted into a suitable vector to transform prokaryotic or eukaryotic host cells.

Further, suitable stimulators or sequences that are involved gene expression may be introduced into the vectors to enable gene expression in the transformed host cells. If desired, a gene of interest may be linked to genes encoding other polypeptides to express it as a fused protein so that it can be purified with greater ease or expressed at higher level. The desired protein can also be excised by applying suitable treatments in the purification step.

It is generally held that eukaryotic genes show polymorphism as is known for the human interferon gene. The polymorphism may cause substitution by one or more amino acids or it may cause changes in base sequences with no change occurring in amino acids.

Even polypeptides having deletion or addition of one or more amino acids within the amino acid sequence of SEQ ID NO: 2, 4 or 15 or having substitution of one or more amino acids may have a cell cycle regulating activity. For example, it is already known that a polypeptide having substitution of cysteine for serine in the human interleukin 2 (IL-2) which is derived from nucleotide alterations has exerted an IL-2 activity (Wang et al., Science 224:1431, 1984). These techniques for preparing modified genes encoding MTf protein are known to the skilled artisan.

In many cases of expression in eukaryotic cells, sugar chains may be added to the protein and the addition of sugar chains can be regulated by substituting one or more amino acids of the protein and even in this case, the chondrogenic differentiation inducing activity may be exhibited. Therefore, genes encoding such polypeptides obtained by using artificial modifications of the gene encoding MTf gene can all be used in the invention, as long as such polypeptides have the chondrogenic differentiation inducing activity.

Expression vectors that can be used include replication origins, selection markers, promoters, RNA splicing sites, polyadenylation signals and so on.

Prokaryotic organisms that can be used as host cells in the expressing system include, for example, Escherichia coli and Bacillus subtilis. Eukaryotic microorganisms that can be used as host cells include, for example, yeasts and myxomycetes. If desired, insect cells such as Sf9 may be used as host cells. Host cells derived from animal cells include, for example, COS cells and CHO cells.

Transformants thus obtained by transforming with the gene encoding MTf protein are cultured to produce proteins. The proteins can be recovered from the transformants or from the cultured medium and be purified. Not only the proteins which are obtained with genes containing nucleotide sequences encoding the amino acid sequences of SEQ ID NO: 2, 4 and 15 but also proteins which are obtained using genes containing altered nucleotide sequences encoding the amino acid sequences having substitution, deletion or addition of one or more amino acids within the amino acid sequences of SEQ ID NO: 2, 4 and 15, or proteins which are obtained using nucleotide sequences that hybridize those altered nucleotide sequences can be used as the chondrogenesis promoter of the invention as long as they have the biological function of MTf protein, namely, the chondrogenic differentiation inducing activity.

Conventional methods for separating and purifying proteins can be employed to separate and purify the

MTf protein. For example, various techniques of chromatography, ultrafiltration, salting-out, dialysis, etc. can appropriately be selected and used in combination.

To use the chondrogenesis stimulator of the invention, the MTf described above may be administered in the form of a protein or it may be used an agent for gene therapy.

Insulin or an insulin-like growth factor has conventionally been known as a chondrocyte differentiating substance. It is interesting to note that the chondrogenesis stimulator of the invention induces chondrogenic differentiation even in the absence of insulin. However, it was found that the effect of the chondrogenesis stimulator is further enhanced in the presence of insulin. Therefore, the desired cartilage repairing action could be further enhanced by using MTf in combination with MTf activating agents such as insulin and an insulin-like growth factor.

When the superntant of a chondrocyte culture was added, marked differentiation of chondrocytes was observed in MTf overexpressing cell lines, suggesting that an MTf activating agent may exist in the conditioned medium of a chondrocyte culture. Therefore, the desired cartilage repairing action could be further potentiated by using MTf in combination with an MTf activating agent.

The MTf activating agent may be obtained by the following methods:

1) purifying from the conditioned medium of a chondrocyte culture system;

2) cloning the cDNA for protein binding to an MTf from a chondrocyte cDNA library; and

3) cloning the cDNA of protein binding to an MTf by the yeast two-hybrid method.

To screen various candidate substances for an MTf activating agent, a method including the following steps can be used:

1) preparing a cell line in which MTf is overexpressed, wherein said cell line retains the ability to differentiate to chondrocytes but hardly differentiate without stimulation;

2) adding candidate substances to the cell line prepared in step 1) and culturing it for a specified period of time; and

3) examining the cell line for induced chondrogenic differentiation and selecting an MTf activating agent from the candidate substances

Thus obtained MTf activating agent can be used as a chondrogenesis stimulator on its own.

It should also be mentioned that chondrogenic differentiation was induced when the soluble MTf, preferably an MTf lacking the GPI anchor region, was expressed in non-MTf-expressing cells. Therefore, the soluble MTf, preferably an MTf lacking the GPI anchor region, can be used as the chondrogenesis regulator.

The chondrogenesis stimulator of the invention can be applied to the following diseases:

1) OA (osteoarthritis);

2) RA (rheumatoid arthritis);

3) injury of articular cartilage due to trauma;

4) maintenance of chondrocyte phenotypes in autologous chondrocyte transplantation;

5) reconstruction of cartilage in the ear, trachea or nose;

6) osteochondritis dissecans;

7) regeneration of intervetebral disk or meniscus;

8) bone fracture;

9) osteogenesis from cartilage.

The chondrogenesis stimulator of the invention is generally useful as an agent for gene therapy having the MTf protein or an MTf mutant (variant) introduced in it. The agent for gene therapy of the invention contains as an active ingredient an expression vector incorporating a DNA coding for any one of the following proteins:

1) a protein having the amino acid sequence of SEQ ID NO: 2;

2) a protein having the amino acid sequence of SEQ ID NO: 4;

3) a protein having the amino acid sequence of SEQ ID NO: 15; 4) a protein demonstrating the MTf activity that has an amino acid sequence encoded by DNA which hybridizes, under stringent conditions, with a DNA encoding the protein of SEQ ID NO: 2, 4 or 15; and

5) a protein which is the same as protein 1), 2), 3) or 4), except that it lacks the GPI anchor region.

DNAs encoding MTf variants can be easily prepared by the skilled artisan using known techniques such as site-directed mutagenesis and PCR [Molecular Cloning: A Laboratory Manual, 2nd ed., Chapter 15, Cold Spring Harbor Laboratory Press (1989), and PCR—A Practical Approach, IRL Press, 200-210 (1991)].

In the present invention, an MTf- or MTf variant expression vector is provided as a DNA to be introduced into cells. These expression vectors can be prepared by linking-DNA encoding an MTf- or MTf variant to an expression vector such as pSG5 (Stratagene). In the next step, the prepared DNA mixture is introduced into cells. Exemplary cells may include bone marrow interstitial cells, fibroblasts, periosteal cells, perichondral cells, synovial cells and dedifferentiated chondrocytes. DNA can be introduced into cells by the calcium phosphate method [Idenshi donyu to hatsugen kaisekiho (Gene Introduction and Methods of Expression and Analysis), ed. by Takashi Yokota and Kenichi Arai, Yodosha, 1994]. Hence, by using the introduced DNA as a medicinal active ingredient, one can prepare an agent for gene therapy which has the chondrogenesis promoting-action. It is thought that, upon administering such agent for gene therapy, MTf or its variant would be expressed at high levels in cells, promoting the action of inducing chondrogenic differentiation in the cells. Therefore, the MTf containing agent of the invention for gene therapy can be used as a therapeutic or preventive of the various diseases listed above.

The agent of the invention for gene therapy can be introduced into cells by either a virus vector based method of gene introduction or a non-viral method of gene introduction [Nikkei Science, April 1994, pp. 20-45, Jikken igaku zokan (Extra Issue of Experimental Medicine), 12 (15) (1994), and Jikken igaku bessatsu (Supplement to Experimental Medicine), “Idenshi chiryo no kiso gijutsu (Basic Technology in Gene Therapy)”, Yodosha (1996)].

In an example of the viral vector based method of gene introduction, DNA encoding an MTf or a variant MTf is inserted into DNA or RNA viruses such as retrovirus, adenovirus, adeno-associated virus, herpes virus, vaccinia virus, poxvirus, poliovirus and Sindbis virus. Non-viral methods of gene introduction include direct intramuscular administration of an expression plasmid (DNA vaccination), liposome method, lipofectin method, microinjection, calcium phosphate method, and electroporation.

In order to ensure that the agent for gene therapy of the invention acts as a practical medicine, two methods may be used, that is, an in vivo method where DNA is directly introduced into the body and an ex vivo method where a certain kind of cells are taken out of a human and DNA is introduced into the cell, which is then put back into the body [Nikkei Science, April 1994, pp. 20-45, Gekkan yakuji (Monthly Yakuji), 36(1), 23-48 (1994), and Jikken igaku zokan (Extra Issue of Experimental Medicine), 12(15) (1994)]. The in vivo method is more preferred.

When administering the agent for gene therapy by the in vivo method, the route of administration should depend on the disease, its severity and other factors. Exemplary methods of administration include intra-articular injection, direct application to a missing part of articular cartilage, implantation (with putty, polylactic acid, etc.) and intra-articular sustained-release agent. Intravenous injection is also possible. For administration by the in vivo method, injections are generally used, with conventional carriers being added as required. Liposomes or membrane fused liposomes may be formulated as suspensions, frozen vesicles, centrifugally concentrated frozen vesicles.

In the case of the GPI anchor free soluble protein (GPI anchor lacking MTf) or the aforementioned MTf activating agent, the above-listed administrating methods may of course be used but the protein per se can be administered by various other methods. Differentiation could also be induced by adding those proteins directly to cartilage precursor cells.

The dose of the chondrogenesis stimulator of the invention is determined specifically by a doctor considering various factors such as the type of the disease to be treated, its severity, the age and body weight of the patient. The GPI anchor free soluble protein (GPI anchor lacking MTf) or the aforementioned MTf activating agent may be administered typically at a dose ranging 1 ng-1000 mg/day, preferably 1 μg-100 mg/day.

The present invention further provides a chondrogenic differentiation suppressing agent containing an MTf antagonist. MTf antagonists include an anti-MTf antibody, antisense DNA based on the nucleotide sequence of MTf, etc.

Antisense DNA is an oligonucleotide or an oligonucletide analog that are capable of hybridizing with an MTf coding nucleic acid.

Antisense DNA has a nucleotide sequence complementary to mRNA and forms a base pair with mRNA, thereby blocking the flow of genetic information to suppress the synthesis of MTf as the final product. The antisense DNA that can be used in the invention is an oligonucleotide capable of specifically hybridizing with a base sequence encoding the amino acid sequence identified by SEQ ID NO: 2, 4 or 15.

The term “oligonucleotide” as used herein means oligonucleotides generated from naturally occurring bases and sugar portions bound by intrinsic phosphodiester bonds, as well as analogs thereof. Therefore, the first group encompassed by this term comprises naturally occurring species or synthetic species that are generated from naturally occurring subunits or homologs thereof. The term “subunit” means a base-sugar combination which links to adjacent subunit by phosphodiester bond or other bond. The second group of oligonucleotides are their analogs that function similar to oligonucleotides but which are composed of residues having non-naturally-occurring moieties. These include oligonucleotides having chemical modifications applied to phosphate groups, sugar portions and 3′- and 5′-ends in order to provide increased stability. Examples are oligophosphorothioate and oligomethylphosphonate in which one of the oxygen atoms in the phosphodiester group between nucleotides is substituted by sulfur and —CH₃, respectively. Phosphodiester bonds may be replaced by other structures which are non-ionic and achiral. Additional oligonucleotide analogs that can be used are species containing modified base forms, that is, purine and pyrimidine in non-naturally-occurring form.

The oligonucleotides to be used in the invention have preferably 5-40 subunits in length, more preferably 8-30 subunits, most preferably 12-30 subunits.

In the present invention, the target portion of mRNA with which oligonucleotides hybridize is preferably a transcription initiation site, a translation initiation site, an intron/exon junction site or a 5′-cap site; considering the secondary structure of mRNA, a site having no steric hindrance should be selected.

In the present invention, peptide nucleic acids (see, for example, Bioconjugate Chem., Vol. 5, No. 1, 1994) may be used in place of oligonucleotides.

In a particularly preferred embodiment of the invention, oligonucleotides or peptide nucleic acids that hybridize with a nucleotide sequence encoding the amino acid sequence identified by SEQ ID NO: 2 and which can inhibit MTf expression is employed.

In the present invention, oligonucleotides can be produced by synthesis methods known in the art, for example, the solid-phase synthesis method using a synthesizer as manufactured by Applied Biosystems. Similar methods can be used to produce oligonucleotide analogs such as phosphorothioate and alkylated derivatives [Akira Murakami et al., “Kinosei antisense DNA no gosei (Chemical Synthesis of Functional Antisense DNA)”, Organic Synthesis Chemistry, 48(3):180-193, 1990].

The MTf antagonist that can be used in the invention is not limited to oligonucleotides of the above-defined antisense DNA providing length. To the extent that production of intrinsic MTf can be suppressed, a longer antisense, preferably an antisense of 500-600 nucleotides in length, may be inserted into a genome to be used for suppressing chondrogenic differentiation (see Example 3).

The anti-MTf antibody to be used in the invention is one that recognizes a peptide having at least five consecutive amino acids in the amino acid sequence identified by SEQ ID NO: 2, 4 or 15; this can be produced using a conventional procedure [see, for example, Shin-seikagaku jikken koza 1 (New Course in Biochemical Experiments 1), Protein I, pp. 389-397, 1992], which comprises immunizing an animal with an antigenic peptide having at least five consecutive amino acids in the amino acid sequence of SEQ ID NO: 2, 4 or 15, isolating the antibody produced in the animal body, and purifying the isolated antibody. The antibody may include a polyclonal and a monoclonal antibody and methods of preparing these antibodies are also known to the skilled artisan.

The following examples are provided to further illustrate the present invention but are in no way to be taken as limiting the invention. Various alterations and modifications can be made by the skilled artisan and are included within the scope of the invention.

### EXAMPLES

Materials and Methods of Experiment

Rabbit Chondrocyte Culture

Chondrocytes were isolated from rabbit costal cartilage using, with necessary modifications, the method of Kato et al. (Kato et al.: J. Cell Biol., vol. 100, pp. 477-485, 1985). Specifically, the resting cartilage of ribs in 4-week old male Japanese albino rabbits (Hiroshima Laboratory Animals) was separated, shredded with a surgical knife and incubated in a Dulbecco's modified Eagle's medium (DMEM, Flow Laboratories) containing 8 mg/mL of actinase E (Kaken Seiyaku) and 5% fetal calf serum for 1 hour and in DMEM containing 0.15% collagenase (Worthington Biochemical) for 3 hours. Cells passing through a 120-μm nyron filter were recovered, seeded in plastic culture dishes (Corning) and grown in an alpha-modified Eagle's medium (α-MEM, Sanko Junyaku) containing 10% fetal calf serum (Mitsubishi Kasei), 50 μg/mL of ascorbic acid, 50 U/mL of G potassium, 60 μg/mL of kanamycin (all being from Meiji Seika) and 250 μg/mL of amphotericin B (ICN Biochemical) (medium A) or in a serum-free DMEM (medium B) at 37° C. in the atmosphere of 5% CO₂ gas.

Mouse Chondrogenic Cell Line ATDC5 Culture

ATDC5 was purchased from Riken Cell Bank (Tsukuba, Japan). The cells were cultured in a 1:1 mixture of Ham F-12 medium (Flow Laboratories) and a Dulbecco's modified Eagle's medium (DMEM, Flow Laboratories) containing 5% fetal calf serum (FCS, Mitsubishi Kasei), 10 μg/mL of human transferrin (Boehringer Mannheim) and 0.3 nmol/mL of sodium selenite (Wako Junyaku) (maintenance medium) at 37° C. in the atmosphere of 5% CO₂ gas. To induce cartilage differentitation, ATDC5 cells were cultured in a medium (differentiation medium) prepared by adding 10 μg/mL of bovine insulin (Sigma) to the maintenance medium.

cDNA Cloning Of Rabbit MTf and Nucleotide Sequencing

Three days after reaching confluence, the rabbit chondrocytes were treated by the guanidine thiocyanate method to extract total RNA. On the basis of the nucleotide sequence of human MTf (Rose, T. M. et al., Pro NAS, 83, 1261-1265, 1986), a primer pair of 5′-GGCTGGAACGTGCCCGTGGGCTA-3′ (forward) (SEQ ID NO: 5) and 5′-GTCCTGGGCCTTGTCCAGCAGTC-3′ (reverse) (SEQ ID NO: 6) was designed and 1.5 kb MTf cDNA fragments were amplified from the total RNA (1 μg) by a reverse transcription polymerase chain reaction (RT-PCR) method. The obtained cDNA fragments were cloned into the SmaI site of pBluescript II SK vectors (Stratagene) and their nucleotide sequence was determined using Sequenase 7-deaza-dGTP DNA sequencing kit (USB). To obtain a full-length cDNA, rapid amplification of cDNA end (RACE) was performed using Marathon cDNA amplification kit (Clontech). Specifically, Marathon cDNA adapters were ligated to the both ends of the double-stranded total cDNA of rabbit chondrocytes and RACE was performed using adapter primers described above and specific primers (5′-AGAGGGACTCCGAGTATCTGGTCTC-3′ (forward) (SEQ ID NO: 7) and 5′-GTCCGGCCCGACACCAACATCTTC-3′ (reverse) (SEQ ID NO: 8) designed from the MTf nucleotide sequence. The amplified cDNA samples were separated in a 4.5% acrylamide gel and the principal cDNA bands were extracted from the gel and the bands were cloned into pBluescript II SK vectors. With the clones used as templates, the nucleotide sequence of full length MTf was determined using Sequenase 7-deaza-dGTP DNA sequencing kit and ABI autosequencer (ABI).

Creating MTf Overexpressing ATDC5 Variant Cells

Rabbit MTf cDNA (Kawamoto T. et al., EJB, 1988) of either full length or a truncated form which had the 28 residues from C-terminal necessary for GPI anchor binding deleted was inserted into pcDNA3.1/Zeo(+) plasmid expression vector (containing a cytomegalovirus very early promoter/enhancer; Invitrogen, San Diego, Calif.). Specifically, an EcoRI-NotI fragment including the full length was excised from a vector and inserted at the EcoRI-NotI site of pcDNA3.1/Zeo(+). To create a variant which lacks the GPI anchor binding site, a fragment was prepared having a stop codon inserted 28 amino acids upstream of the C-terminal and after confirming its sequence, the fragment was inserted at the EcoRI-NotI site of pcDNA3.1/Zeo(+).

In these ways, there were prepared a plasmid having a full length MTf cDNA (MTf Full) as an insert and a plasmid having GPI anchor-lacking MTf cDNA (MTf(−)GPI) as an insert; the two plasmids (pMTf Full and pMTf(−)GPI) were each transfected to ATDC5 cells (Riken, Tsukuba, Japan) using SuperFect Transfection Reagent (QIAGEN). By selection with Zeocin (Invitrogen), stable transformants were prepared.

Specifically, 2×10⁵ ATDC5 cells were seeded in 10-cm culture dishes. On the next day, 2 μg each of the plasmid DNAs to be introduced (pMTf Full and pMTf(−)GPI) and about 40 μL of SuperFect Transfection Reagent in solution were individually dissolved in a serum-free medium and stored until use, when they were rapidly mixed together and added to ATDC5 cells washed with a serum-free medium. After incubation at 37° C. for 1 hour in the atmosphere of 5% CO₂ gas, a serum-supplemented medium was added and cultivation was conducted for an additional day. A control group was prepared by transfecting only the vector.

One day after the transfection, selection was started in a serum-supplemented medium containing 50 μg/mL of Zeocin and cell culture was continued for 2 weeks with medium change effected on every third day. As a result, there were obtained ATDC5 variant cell lines that would assure stable expression of MTf Full and MTf(−)GPI and these variant cell lines were subcultured in a serum-supplemented medium containing 50 μg/mL of Zeocin.

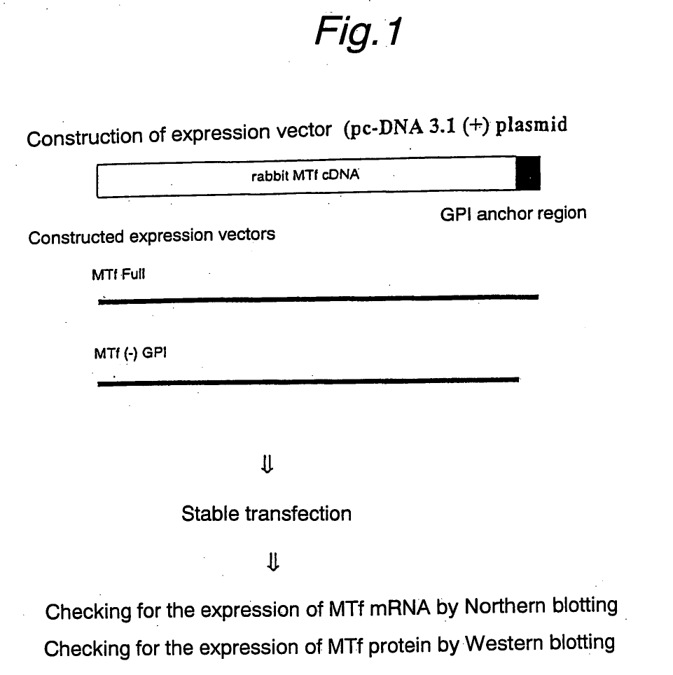

A scheme of the procedure of creating MTf overexpressing ATDC5 variant cells is shown in FIG. 1.

Expression of Rabbit MTf Gene in ATDC5 Variant Cell Lines

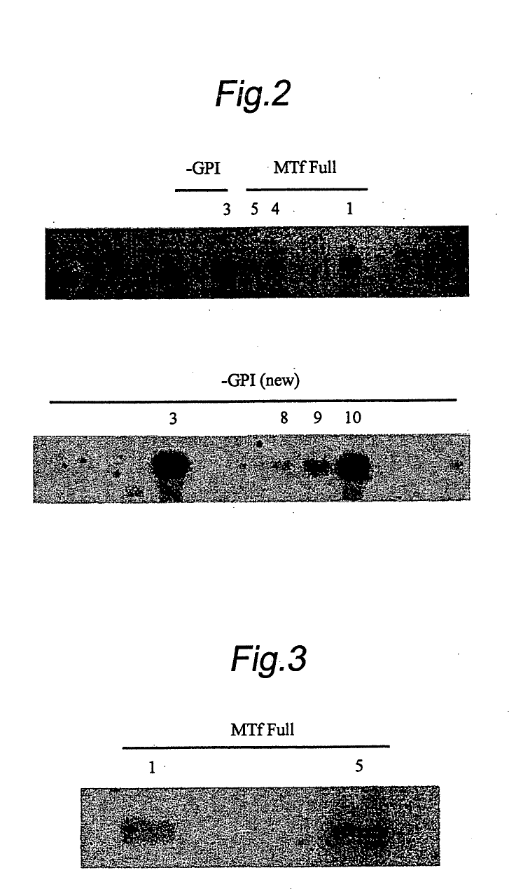

Expression of rabbit MTf gene in ATDC5 variant cell lines was confirmed by Northern blotting. Specifically, total RNA was prepared from the ATDC5 variant cell lines by the guanidine thiocyanate method; 10 μg of the total RNA was electrophoresed on a 1% agarose gel containing 2.2 mol/L of formaldehyde and transferred onto Hybond-N membrane (Amersham). The membrane was hybridized with a ³²P labeled 2.2 kb rabbit MTf cDNA probe at 42° C. for 16 hours. After washing the membrane, a BioMax X-ray film (Kodak) was exposed to the membrane at −80° C. to detect signals. The result is shown in FIG. 2.

In the MTf Full cell line, it was found that the rabbit MTf gene have been expressed strongly in clone Nos. 1, 4 and 5.

In the MTf(−)GPI cell line, it was found that the rabbit MTf gene have been expressed strongly in clone Nos. 3, 3N, 8, 9 and 10. In the Examples, (−)GPI-3 was used.

Expression of Rabbit MTf Protein in ATDC5 Variant Cell Lines

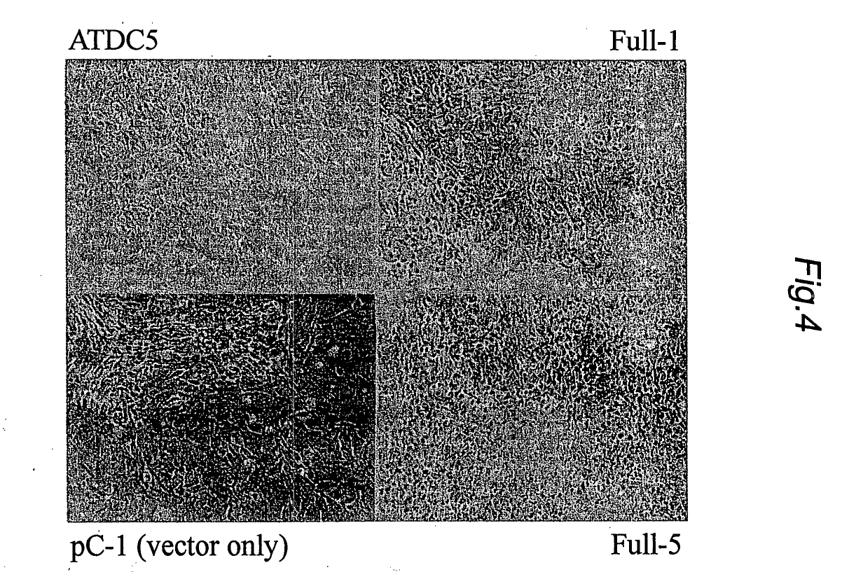

Expression of rabbit MTf protein in ATDC5 variant cell lines was confirmed by Western blotting. Specifically, membrane fraction protein was prepared from the ATDC5 variant cell lines, subjected to SDS-PAGE at 10 μg/lane, and transferred to a polyvinylidene difluoride membrane (Milipore). After the transfer, the membrane was blocked with 4% skimmed milk and reacted with anti-MTf serum [1:500 dilution; Eur. J. Biochem, 256, 503-509 (1988)] at 4° C. for 14 hours, then reacted with ¹²⁵I sheep anti-mouse IgG(Fab′)2 fragment (Amersham) at room temperature for 2 hours. The membrane was washed and a BioMax X-ray film was exposed to the membrane at −80° C. for analysis. The result is shown in FIG. 3.

In the MTf Full cell line, it was found that the rabbit MTf protein have been expressed strongly in clone Nos. 1 and 5. These clones were named MTf overexpressing cell lines (Full-1 and Full-S).

### Example 1

### Chondrogenic Differentiation in MTf Overexpressing Cell Lines

The MTf overexpressed cell lines (4.0×10⁴ cells) were seeded in 6-multiwell plates and cultured in a maintenance medium at 37° C. in a 5% CO₂ gas phase.

The MTf overexpressing cell lines to be investigated were Full-1 and Full-5 which were found to have expressed both the MTf gene and protein. The MTf(−)GPI cell line to be investigated was GPI-3 which was found to have expressed the MTf gene.

As control cells, ATDC5 cells and pC-1 (vector alone) were prepared in the same manner as described above and their morphological features were examined under a microscope. Cell morphology was examined with an Olympus phase-contrast microscope. Two microscopic fields were taken for each culture system and at least 200 cells were counted to calculate the proportion of round cells.

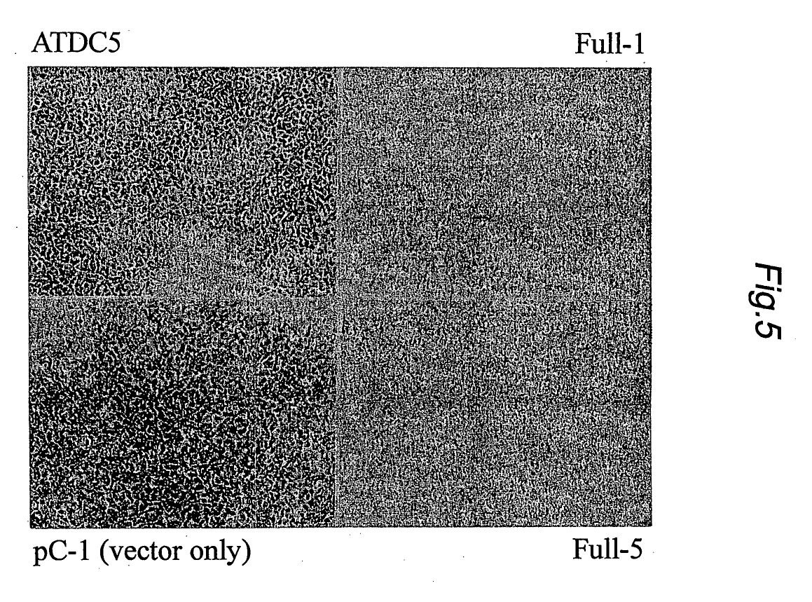

In the absence of insulin, the control cells (pC-1) did not differentiate to chondrocytes; on the other hand, the MTf overexpressing cell lines (Full-1 and Full-5) and the MTf(−)GPI cell line [(−)GPI-3] started to differentiate within 20 days and 29 days after seeding, almost all regions of the cells had differentiated to chondrocytes (FIG. 4).

The similar test was conducted in the presence of insulin (10 μg/mL) (insulin was added at day 0). The result was the same as in the absence of insulin (FIG. 5), except that, in the presence of insulin, further differentiation was induced in the MTf overexpressing cell lines, namely, more cells “rounded” like chondrocytes than in the absence of insulin.

The above results show the effectiveness of MTf in inducing chondrogenic differentiation, which was exhibited even in the absence of insulin.

### Example 2

### Effect of Adding the Conditioned Medium of Rabbit Chondrocyte Culture

Resting rabbit chondrocytes (1×10⁶ cells) were seeded in 10-cm culture dishes and cultured in medium A (10 mL) at 37° C. in the atmosphere of 5% CO₂ gas. Two days after confluence, medium A was replaced by serum-free medium B (5 mL). After 24 hours of cell culture, the conditioned medium (CM) was recovered and subjected to an experiment. After the recovery, CM was supplemented with fetal calf serum at a concentration of 5%.

MTf overexpressing cell line (Full-5) and the control cell line (pC-1) were seeded at 8.0×10⁴ cells in 6-multiwell plates and cultured in a maintenance medium at 37° C. in the atmosphere of 5% CO₂ gas. Three days after confluence (day 7), the previously recovered CM was added to give a concentration of 60% in the overall liquid culture; at the same time, 10 μg/mL of bovine insulin was added. Cultivation was continued for additional 48 hours at 37° C. in a 5% CO₂ gas phase.

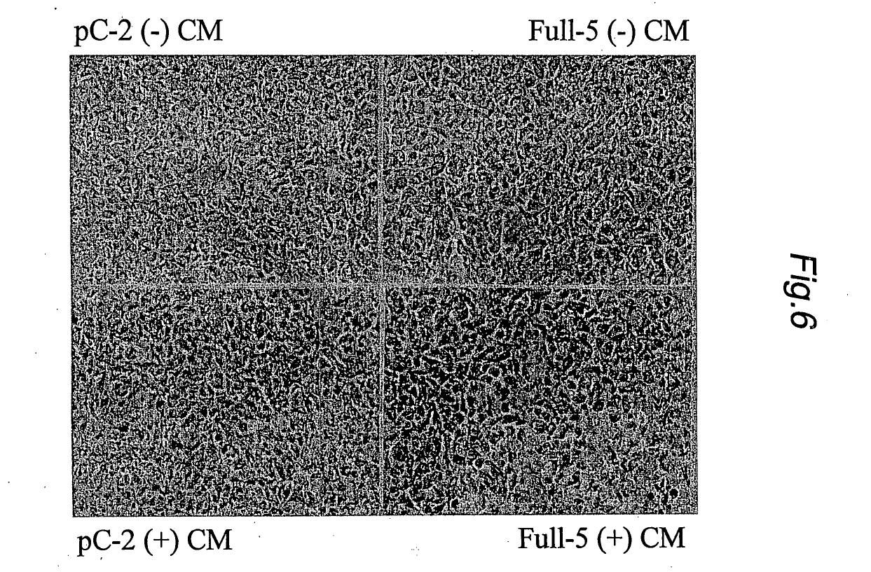

Forty-eight hours after the addition of CM, almost all cells of the MTf overexpressing cell line to which CM was added [Full-5(+)CM] differentiated to chondrocytes which synthesized active substrate and which resembled paving stones. The cells of the MTf expressing cell line to which no CM was added [Full-5(−)CM] had almost the same morphology as the control cell lines in which only a vector was expressed [pC-2(+)CM and (−)CM]. The cell lines in which only a vector was expressed had no visible induced differentiation to chondrocytes due to the addition of CM (FIG. 6).

These results show the presence of an MTf activating agent in CM.

### Example 3

### Chondrogenic Differentiation Due to Overexpression of Antisense MTf RNA

ATDC5 variant cell lines (A-01, A-05, A-08, A-09, A-11, A-12, A-23 and A-24) in which mouse antisense MTf RNA was overexpressed were prepared by the similar method used in preparing the ATDC5 variant cell lines in which MTf was forcibly expressed. The mouse antisense MTf RNA was prepared as follows: cDNA fragments of mouse MTf were amplified by PCR (using primers 5′-GGTGTGTTGAGGGGCGTGGACTCT-3′ (SEQ ID NO: 9) and 5′-TCACCAACGGCTTTGAGCACATCAC-3′ (SEQ ID NO: 10), inserted into pGEM-T Easy Vector (Promega), excised with ApaI-NotI and inserted into pcDNA3.1/Zeo(+) at ApaI-NotI site (i.e., inserted in reverse direction). The sequence of the inserted portion is identified by SEQ ID NO: 11.

A control group was also prepared by transfecting only the vector (pC-1).

The expression of mouse MTf antisense was examined by Northern blotting using the above-mentioned ApaI-NotI fragment as a probe.

A criterion for the suppression of chondrogenic differentiation was the suppression of synthesis of a cartilage proteoglycan, aggrecan, and a test was conducted by RT-PCR Southern blotting both in the presence of insulin (added in an amount of 10 μg/mL after day 4) and in its absence. Specifically, total RNA was extracted from the cells of each clone by the guanidine thiocyanate method; single-stranded cDNA was synthesized from the extracted total RNA (1 μg) using SUPERSCRIPT pre-amplification system kit (Life Technologies); using the cDNA as a template, PCR was performed on the aggrecan gene with a pair of primers 5′-TGCTACTTCATCGACCC-3′ (forward) (SEQ ID NO: 12) and 5′-AAAGACCTCCCCTCCATCT-3′ (reverse) (SEQ ID NO: 13); the PCR reaction mixture was electrophoresed on a 1% agarose gel and transferred onto Hybond-N membrane (Amersham). The membrane was hybridized with a ³²P labeled antisense MTf probe and mouse aggrecan cDNA probe at 42° C. for 16 hours. The membrane was washed with 0.2×SSC whih contains 0.5% SDA and a BioMax X-ray film (Kodak) was exposed to the membrane at −80° C. to detect signals.

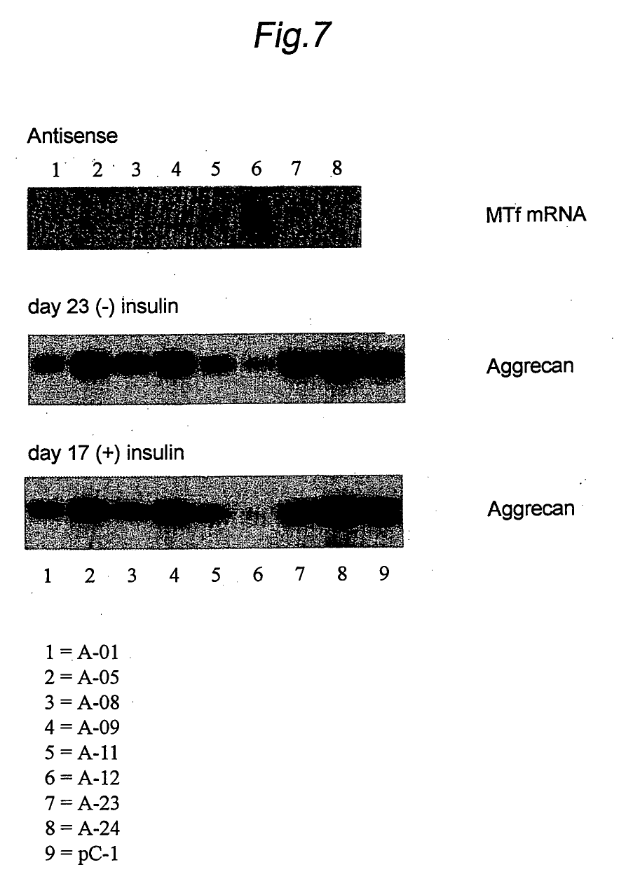

The result is shown in FIG. 7. Antisense MTf RNA was expressed most strongly in variant cell line A-12 (lane 6), followed by A-11 (lane 5) in strength.

Correspondingly, expression of the aggrecan gene was suppressed most effectively in variant cell line A-12 (lane 6), followed by A-11 (lane 5) in effectiveness. While expression of the aggrecan gene was suppressed in the absence of insulin, it was more effectively suppressed in the presence of insulin.

1. A method for stimulating cartilage formation comprising administering to a patient in need thereof an effective amount of membrane-bound transferrin-like protein (MTf).

2. The method according to claim 1 wherein the MTf is selected from the group consisting of, human p97 protein and a protein demonstrating MTf activity that has an amino acid sequence encoded by DNA which hybridizes, under stringent conditions that consist of hybridization at 68° C. in a solution containing 6×SSC, 0.5% SDS, 10 mM EDTA, 5× Denhardt's solution and 10 mg/ml of denatured salmon sperm DNA, with a DNA coding for the human p97 protein.

3. A method for stimulating chondrogenesis comprising administering to a patient in need thereof an effective amount of MTf.

4. The method according to claim 3 wherein the MTf is selected from the group consisting of human p97 protein, and a protein demonstrating MTf activity that has an amino acid sequence encoded by DNA which hybridizes, under stringent conditions that consist of hybridization at 68° C. in a solution containing 6×SSC, 0.5% SDS, 10 mM EDTA, 5× Denhardt's solution and 10 mg/ml of denatured salmon sperm DNA, with a DNA coding for the human p97 protein.

5. The method according to claim 3 wherein the patient in need thereof is suffering from a bone disease selected from the group consisting of the following diseases in which chondrogenic differentiation is involved: osteoarthritis; rheumatoid arthritis; injury of articular cartilage due to trauma; maintenance of chondrocyte phenotypes in autologous chondrocyte transplantation; reconstruction of cartilage in the ear, trachea or nose; osteochondritis dissecans; regeneration of intervetebral disk or meniscus; bone fracture; and osteogenesis from cartilage.

6. The method according to claim 3 wherein the MTf is selected from the following:
a. a protein having the amino acid sequence of SEQ ID NO: 4; and
b. a protein demonstrating the MTf activity that has an amino acid sequence encoded by DNA which hybridizes, under stringent conditions that consist of hybridization at 68° C. in a solution containing 6×SSC, 0.5% SDS, 10 mM EDTA, 5× Denhardt's solution and 10 mg/ml of denatured salmon sperm DNA, with a DNA encoding the protein of SEQ ID NO: 4.

7. The method according to claim 3 wherein the chondrogenesis stimulator is used in combination with insulin.

8. The method according to claim 3 wherein the MTf is soluble MTf.

9. The method according to claim 8 wherein the soluble MTf lacks the GPI anchor region.


SEQUENCE LISTING (15 sequences)

SEQ ID NO: 1 - DNA, 2388 bp, Oryctolagus cuniculus
gccgccgctcactcgttcgcactcggactcagacccagtccgaccccctggactgcgccatgcggtgccgaagcgcggctatgtggatcttcctggccctgcgcaccgcactcggcagcgtggaggtgcggtggtgcaccgcgtccgagcccgagcagcagaagtgcgaggacatgagccaggccttccgcgaagccggcctccagcccgccctgctgtgcgtgcagggcacctcggccgaccactgcgtccagctcatcgcggcccacgaggccgacgccatcactctggacggaggagccatttacgaggcggggaaggaacacggcctgaagcccgtggtgggcgaagtgtatgaccaagaggtgggcacctcctactacgctgtggccgtggtcaagaggagctccaacgtgaccatcaacaccctgagaggcgtgaagtcctgccacacgggcatcaaccgcacggtgggctggaacgtgcctgtgggctacctggtggacagcggccgcctctcagtgatgggctgtgacgtgctcaaagcggtcagcgagtacttcgggggcagctgcgtccctggggcaggagagaccagatactcggagtccctctgtcgcctctgccggggcgacacctccggggagggggtgtgtgacaagagccccctggagcggtactacgactacagcggggccttccggtgcctggcagaaggcgcaggggacgtggcctttgtgaagcacagcacggtgctggagaacacggatgggagaacactgccctcctggggccacatgctgatgtcacgggactttgagctgctgtgccgggacggcagccgggccagcgtcaccgagtggcagcactgccacctggcccgggtgcccgcccacgccgtggtggtccgggccgacaccgacgcaggcctcatcttccggcttctcaatgagggccagcggctgttcagccacgagggcagcagcttccagatgttcagctcggaggcctacggccagaagaacctgctgttcaaagactccacgctggagctggtgcccatcgccacacagacctacgaggcctggctgggccccgagtacctgcacgccatgaagggtctgctctgtgaccccaaccggctgcccccatacctgcgctggtgcgtgctgtccacccccgagatccagaagtgtggagacatggccgtggccttcagccggcagaggctcaagccggagatccagtgtgtctcggcggagtccccccagcactgcatggagcagatccaggctgggcacatcgatgctgtgaccctgaacggggaggacattcacacagcggggaagacttatgggctgatcccggctgccggggagctgtatgccgcggacgacaggagtaactcgtacttcgtggtggccgtggtgaagcgagacagcgcctacgccttcaccgtggacgagctgcgcgggaagcgctcctgccaccccggcttcggcagcccggccggctgggacgtcccggtgggcgccctcatccactggggctacatccggcccaggaactgcgacgtcctcacagcggtgggtcagttcttcaacgccagctgtgtgccggtgaacaaccccaagaagtacccctcctcgctgtgcgcactctgcgtgggtgacgagcagggccgcaacaagtgcactggcaacagccaggagcggtactatggcgacagtggcgccttcaggtgcctggtggagggtgcaggggacgtggccttcgtcaagcacacgaccatctttgacaacacaaatggccacaatcccgagccgtgggctgcccatctgaggagccaggactacgagctgctgtgccccaacggcgcgcgagctgaggcgcaccagtttgccgcctgcaacctggcccagattccgtcccacgccgtcatggtgcggcccgacaccaacatcttcaccgtttacggactgctggacaaggcccaggacctgtttggagacgaccacaacaagaacgggttcaagatgttcgactcctccagctaccacggccgagacctgctcttcaaggacgccacggtgcgcgctgtgcctgtgggcgagaggaccacctaccaggactggctggggccggactacgtggcggctctggaagggatgcagtcacagcggtgctcaggggcagccgtcggcgcccccggggcctcgctgctgccgctgctgcccctggctgcgggcctcctgctgtcttcgctctgagagcagccccgggcagcctcggccccggcaggggagcctgcgcggaagcttcctgaacgagcccgcgccctggctggatgtggttacctcggcgagccgcggggccgcgcttcccccg

SEQ ID NO: 2 - protein, 736 aa, Oryctolagus cuniculus
MRCRSAAMWIFLALRTALGSVEVRWCTASEPEQQKCEDMSQAFREAGLQPALLCVQGTSADHCVQLIAAHEADAITLDGGAIYEAGKEHGLKPVVGEVYDQEVGTSYYAVAVVKRSSNVTINTLRGVKSCHTGINRTVGWNVPVGYLVDSGRLSVMGCDVLKAVSEYFGGSCVPGAGETRYSESLCRLCRGDTSGEGVCDKSPLERYYDYSGAFRCLAEGAGDVAFVKHSTVLENTDGRTLPSWGHMLMSRDFELLCRDGSRASVTEWQHCHLARVPAHAVVVRADTDAGLIFRLLNEGQRLFSHEGSSFQMFSSEAYGQKNLLFKDSTLELVPIATQTYEAWLGPEYLHAMKGLLCDPNRLPPYLRWCVLSTPEIQKCGDMAVAFSRQRLKPEIQCVSAESPQHCMEQIQAGHIDAVTLNGEDIHTAGKTYGLIPAAGELYAADDRSNSYFVVAVVKRDSAYAFTVDELRGKRSCHPGFGSPAGWDVPVGALIHWGYIRPRNCDVLTAVGQFFNASCVPVNNPKKYPSSLCALCVGDEQGRNKCTGNSQERYYGDSGAFRCLVEGAGDVAFVKHTTIFDNTNGHNPEPWAAHLRSQDYELLCPNGARAEAHQFAACNLAQIPSHAVMVRPDTNIFTVYGLLDKAQDLFGDDHNKNGFKMFDSSSYHGRDLLFKDATVRAVPVGERTTYQDWLGPDYVAALEGMQSQRCSGAAVGAPGASLLPLLPLAAGLLLSSL

SEQ ID NO: 3 - DNA, 2368 bp, Homo sapiens
gcggacttcctcggacccggacccagccccagcccggccccagccagccccgacggcgccatgcggggtccgagcggggctctgtggctgctcctggctctgcgcaccgtgctcggaggcatggaggtgcggtggtgcgccacctcggacccagagcagcacaagtgcggcaacatgagcgaggccttccgggaagcgggcatccagccctccctcctctgcgtccggggcacctccgccgaccactgcgtccagctcatcgcggcccaggaggctgacgccatcactctggatggaggagccatctatgaggcgggaaaggagcacggcctgaagccggtggtgggcgaagtgtacgatcaagaggtcggtacctcctattacgccgtggctgtggtcaggaggagctcccatgtgaccattgacaccctgaaaggcgtgaagtcctgccacacgggcatcaatcgcacagtgggctggaacgtgcccgtgggctacctggtggagagcggccgcctctcggtgatgggctgcgatgtactcaaagctgtcagcgactattttgggggcagctgcgtcccgggggcaggagagaccagttactctgagtccctctgtcgcctctgcaggggtgacagctctggggaaggggtgtgtgacaagagccccctggagagatactacgactacagcggggccttccggtgcctggcggaaggggcaggggacgtggcttttgtgaagcacagcacggtactggagaacacggatgggaagacgcttccctcctggggccaggccctgctgtcacaggacttcgagctgctgtgccgggatggtagccgggccgatgtcaccgagtggaggcagtgccatctggcccgggtgcctgctcacgccgtggtggtccgggccgacacagatgggggcctcatcttccggctgctcaacgaaggccagcgtctgttcagccacgagggcagcagcttccagatgttcagctctgaggcctatggccagaaggatctactcttcaaagactctacctcggagcttgtgcccatcgccacacagacctatgaggcgtggctgggccatgagtacctgcacgccatgaagggtctgctctgtgaccccaaccggctgcccccctacctgcgctggtgtgtgctctccactcccgagatccagaagtgtggagacatggccgtggccttccgccggcagcgcctcaagccagagatccagtgcgtgtcagccaagtccccccaacactgcatggagcggatccaggctgagcaggtcgacgctgtgaccctaagtggcgaggacatttacacggcggggaagaagtacggcctggttcccgcagccggcgagcactatgccccggaagacagcagcaactcgtactacgtggtggccgtggtgagacgggacagctcccacgccttcaccttggatgagcttcggggcaagcgctcctgccacgccggtttcggcagccctgcaggctgggatgtccccgtgggtgcccttattcagagaggcttcatccggcccaaggactgtgacgtcctcacagcagtgagcgagttcttcaatgccagctgcgtgcccgtgaacaaccccaagaactacccctcctcgctgtgtgcactgtgcgtgggggacgagcagggccgcaacaagtgtgtgggcaacagccaggagcggtattacggctaccgcggcgccttcaggtgcctggtggagaatgcgggtgacgttgccttcgtcaggcacacaaccgtctttgacaacacaaacggccacaattccgagccctgggctgctgagctcaggtcagaggactatgaactgctgtgccccaacggggcccgagccgaggtgtcccagtttgcagcctgcaacctggcacagataccaccccacgccgtgatggtccggcccgacaccaacatcttcaccgtgtatggactgctggacaaggcccaggacctgtttggagacgaccacaataagaacgggttcaaaatgttcgactcctccaactatcatggccaagacctgcttttcaaggatgccaccgtccgggcggtgcctgtcggagagaaaaccacctaccgcggctggctggggctggactacgtggcggcgctggaagggatgtcgtctcagcagtgctcgggcgcagcggccccggcgcccggggcgcccctgctcccgctgctgctgcccgccctcgccgcccgcctgctcccgcccgccctctgagcccggccgccccgccccagagctccgatgcccgcccggggagtttccgcggcggcctctcgcgctgcggaatccagaaggaagctcgcga

SEQ ID NO: 4 - protein, 738 aa, Homo sapiens
MRGPSGALWLLLALRTVLGGMEVRWCATSDPEQHKCGNMSEAFREAGIQPSLLCVRGTSADHCVQLIAAQEADAITLDGGAIYEAGKEHGLKPVVGEVYDQEVGTSYYAVAVVRRSSHVTIDTLKGVKSCHTGINRTVGWNVPVGYLVESGRLSVMGCDVLKAVSDYFGGSCVPGAGETSYSESLCRLCRGDSSGEGVCDKSPLERYYDYSGAFRCLAEGAGDVAFVKHSTVLENTDGKTLPSWGQALLSQDFELLCRDGSRADVTEWRQCHLARVPAHAVVVRADTDGGLIFRLLNEGQRLFSHEGSSFQMFSSEAYGQKDLLFKDSTSELVPIATQTYEAWLGHEYLHAMKGLLCDPNRLPPYLRWCVLSTPEIQKCGDMAVAFRRQRLKPEIQCVSAKSPQHCMERIQAEQVDAVTLSGEDIYTAGKKYGLVPAAGEHYAPEDSSNSYYVVAVVRRDSSHAFTLDELRGKRSCHAGFGSPAGWDVPVGALIQRGFIRPKDCDVLTAVSEFFNASCVPVNNPKNYPSSLCALCVGDEQGRNKCVGNSQERYYGYRGAFRCLVENAGDVAFVRHTTVFDNTNGHNSEPWAAELRSEDYELLCPNGARAEVSQFAACNLAQIPPHAVMVRPDTNIFTVYGLLDKAQDLFGDDHNKNGFKMFDSSNYHGQDLLFKDATVRAVPVGEKTTYRGWLGLDYVAALEGMSSQQCSGAAAPAPGAPLLPLLLPALAARLLPPAL

SEQ ID NO: 5 - DNA, 23 bp, Artificial Sequence
  synthetic
ggctggaacgtgcccgtgggcta

SEQ ID NO: 6 - DNA, 23 bp, Artificial Sequence
  synthetic
gtcctgggccttgtccagcagtc

SEQ ID NO: 7 - DNA, 25 bp, Artificial Sequence
  synthetic
agagggactccgagtatctggtctc

SEQ ID NO: 8 - DNA, 24 bp, Artificial Sequence
  synthetic
gtccggcccgacaccaacatcttc

SEQ ID NO: 9 - DNA, 24 bp, Artificial Sequence
  synthetic
ggtgtgttgaggggcgtggactct

SEQ ID NO: 10 - DNA, 25 bp, Artificial Sequence
  synthetic
tcaccaacggctttgagcacatcac

SEQ ID NO: 11 - DNA, 614 bp, Mus sp.
tcaccaacggctttgagcacatcacagcccatcactgacagatggccgctctctacgaggtaaccgacaggcacgttccagcccacagtccggttaatgcctgtgtggcaggacttgacgcccttcagggtgttgatggtaacattggaattcctcctgaccacagccacggcataataggaagtcccaatgtcttggtcatagacttcccccaccactggcttcaggccgtgctccttccctgcctcatagatggcccctccatccagggtgatggcatctgctttttgttccttgatgagctggacacagtggtcagcggagttgccctggacgcagagaagggaaggacgaatgccagctccctggaaggcctcgctcatgtctttgcacttctgctgctctgcgtctgagatggtacaccactgcacctccatcacacagacgacagtgcgcagggacaggagtagccaaaaagtcacgctcaggagcctcatggcaacgttgggttggctggggtgctggcgggtctgtcctggcttcctcttccctggtctctctggccttcactatttaagcgcagcccggggagagtccacgcccctcaacacacc

SEQ ID NO: 12 - DNA, 17 bp, Artificial Sequence
  synthetic
tgctacttcatcgaccc

SEQ ID NO: 13 - DNA, 19 bp, Artificial Sequence
  synthetic
aaagacctcccctccatct

SEQ ID NO: 14 - DNA, 4158 bp, Mus sp.
ggtgtgttgaggggcgtggactctccccgggctgcgcttaaatagtgaaggccagagagaccagggaagaggaagccaggacagacccgccagcaccccagccaacccaacgttgccatgaggctcctgagcgtgactttttggctactcctgtccctgcgcactgtcgtctgtgtgatggaggtgcagtggtgtaccatctcagacgcagagcagcagaagtgcaaagacatgagcgaggccttccagggagctggcattcgtccttcccttctctgcgtccagggcaactccgctgaccactgtgtccagctcatcaaggaacaaaaagcagatgccatcaccctggatggaggggccatctatgaggcagggaaggagcacggcctgaagccagtggtgggggaagtctatgaccaagacattgggacttcctattatgccgtggctgtggtcaggaggaattccaatgttaccatcaacaccctgaagggcgtcaagtcctgccacacaggcattaaccggactgtgggctggaacgtgcctgtcggttacctcgtagagagcggccatctgtcagtgatgggctgtgatgtgctcaaagccgttggtgattattttggaggcagctgtgtccctggaacaggagaaaccagccattccgagtccctctgtcgcctctgccgtggcgactcttctgggcacaatgtgtgtgacaagagtcccctagagagatactacgactacagtggagccttccggtgcctggcggaaggagccggtgacgtggccttcgtgaagcacagcacagtgctggaaaatactgatggaaacaccctgccttcctggggcaagtccctgatgtcagaggacttccagctactatgcagggatggcagccgagccgacatcactgagtggagacgttgccacctggccaaggtgcctgctcatgctgtggtggtcaggggtgacatggatggcggtctcatattccaactgctcaacgaaggccagcttctgttcagccacgaagacagcagcttccagatgttcagctccaaagcctacagccagaagaacttgctgttcaaagactccaccttggagcttgtgcccattgccacacagaactacgaggcctggctgggccaggaatacctgcaggccatgaaggggctcctctgtgatcccaaccggctgccccactacctgcgctggtgtgtgctgtcagcgcccgagatccagaagtgtggagatatggctgtggccttcagccgccagaatctcaagccggaaattcagtgtgtgtcggccgagtcccctgagcactgcatggagcagatccaggctgggcacactgacgctgtgactctgaggggcgaggacatttacagggcaggaaaggtgtacggcctggttccggcggccggggagctgtatgctgaggaggacaggagcaattcctactttgtggtggctgtggcaagaagggacagctcctactccttcaccctggacgagcttcgcggcaagcgttcctgccacccctacttgggcagcccagcgggctgggaggtgcccatcggctccctcatccagcggggcttcatccggcccaaggactgtgatgtcctcacagcggtgagccagttcttcaatgccagctgcgtgcctgtcaacaaccctaagaactacccttccgcactatgtgcgctctgcgtgggagacgagaagggccgcaacaaatgtgtggggagcagccaggagagatactacggctacagcggggccttcaggtgccttgtggagcatgcaggggacgtggctttcgtcaagcacacgactgtctttgagaacacaaatggtcacaatcctgagccttgggcttctcacctcaggtggcaagactatgaactactgtgccccaatggggcacgggctgaggtagaccagttccaagcttgcaacctggcacaaatgccatcccacgctgtcatggtccgtccagacaccaacatcttcactgtgtatggacttctggacaaggcccaggacctgtttggagacgaccataacaagaacggtttccaaatgtttgactcctccaaatatcacagccaagacctgcttttcaaagatgctacagtccgagcggtgccagtccgggagaaaaccacatacctggactggctgggtcctgactatgtggttgcgctggaggggatgttgtctcagcagtgctccggtgcaggggccgcggtgcagcgagtccccctgctggccctgctcctgctgaccctggctgcaggcctccttcctcgcgttctctgaagaccgctgcttcaggccacgcccagagcagggaaagctacagagctcaaccggaagaaaccaggacatcagctaaccctgcaggagagcgcggggcgggatgaggagaggcaaggtgagaactcacacacacacacaagcctccgaggtgcgattctaacccaaagagaaatttctagaatcaggatgattgttaaggccaagtcttcccacttgctggagccctcaatacctgaggcgactggcgagtagcccagtcactcctcccacaccggtggcgccagcagcgaacctgtgcctcccacctggagcctcctggctggctggggtggttaagggggggggggggagagtgaagatgctggttgccatggcaaccgtggagcagcttccagcctctgtaccggccacctggtgagatgccaaggaaggagcacaccaccaacctagggaacctgtgcgacacactaccacccagcagcccctgctctcgctgccccaccgctctctcctatgggcacttgtccaccaaggccacaccgtcggaggggcaaggctgctgagcacatcagccttctgatgtgacaccaaccaaggagcccagccctctggacagcaagattttgctagactgggatgggaggaaggccagagctgtactgtggggatgaagtcctccaaaaccctcagaggaaggaagtgcccccaccttcccattaagaatgttagtgtgtgagaaacttgatgcagggtggaaactatcctgtttaacggctcccgtggcaagcaggacttgcgctgtctgcgctgcctggacctcactgcacaatgaaactgttgccgagattctattgtttgctctcctggtctcagtctcaacattagttttctccctgccttcatataccccttcccacatcaccacgcaagcacgcacgcgcacacgcacacgcacacaccttatccgtgtgaacatatctgaacatatctgcttgtctgaagaagtaggagctaacccaaaataacttcctgtcatgagctgggccttgggatataccacgagccaggggattggggagagccctgtcttcccttcaccctgcacctgttgggcagttgcatctttcgagaggatccctggttctctcgaactgtgagagccaaggcctaggctgccattttgccattgttctctcgagaaccagaaaaagttttccaaagctaccagctcttaccccagatcttgttcccttaaaaaaaagtaataaataaaaaggagaagaaacaggagcaaacagccatcgtcagcacactggaagcagcgtgggccgggagctatttgtgtcttggtctgtgtggggggcctcagatcccaatgacaggccaggttcccagtggctcgcccccacctgtgggcgacgacgggacagatcctttccatggctcaccagtagagaaggtcctggcagtgtcccagccagagtcacacaatcctgaggaaaatcggtcaccatggtgcttgggagagcaagcccctcctcctcccagtacacagccatccattcttctctgagctggggacttcacagtgagaagtgtactctgtgtgggcgactgtgctgcccaaagtgtgatgtctgtgccgtgtgcctttcaggtgtgactttgaagagcgttgtgtaaatgacgtctgattgccatgggccactgctgtgtttgtgctaaagaaagacattggtttctttttaaaataaagccatatatccctgcatacgcagaggcttggatcctggtggaaaaaaaaaaaaaaaaaaaaaaaaaaaa

SEQ ID NO: 15 - protein, 738 aa, Mus sp. [sequence omitted]